(19) United States
(12) Patent Application Publication
(10) Pub. No.: US 2023/0172290 A1
(43) Pub. Date: Jun. 8, 2023

(54) HALF MASK FOR THE FACE

(71) Applicant: DIVIS GMBH, Wiehl (DE)

(72) Inventor: Vanessa KLAWUNDER-RYLL, Reichshof (DE)

(21) Appl. No.: 18/074,607

(22) Filed: Dec. 5, 2022

(30) Foreign Application Priority Data
Dec. 6, 2021 (DE) 102021006010.5

(51) Int. Cl. A41D 13/11; A62B 23/02; A62B 9/00; A61L 9/20

(52) U.S. Cl. CPC A41D 13/1107; A62B 23/02; A62B 9/006; A61L 9/20; A61L 2209/12; A61L 2209/111; A61L 2209/14

(57) ABSTRACT

A half mask for the face, including one or more covering areas for covering one or both respiratory openings of the person wearing the half mask and one or more fastening areas for fastening the half mask to its wearer, particularly to one or more of their head, neck, and throat, one or more UV-C disinfection units for disinfecting the inhaled and exhaled air and one or more devices for fitting the half mask tightly around the face of its wearer are provided.

### FIELD OF THE INVENTION

The invention relates to a half mask for the face, comprising at least one area covering one or both respiratory openings of the wearer of the half mask and at least one area for fastening the half mask to its wearer, particularly to one or more of their head, neck, and throat.

### BACKGROUND OF THE INVENTION

Such half masks are known in the prior art. Respiratory masks of all types have always been deemed a method of choice for preventing undesired matter of all sizes from penetrating a person's respiratory tract. And such a mask can also keep particles from being released into the surrounding air during exhalation, depending on the mask's design. One normally tries to achieve this by using a more or less coarse filter material (such as woven or braided fabric) to filter out substances from a certain particle size by means of this filter material, thus restricting or preventing particles from a certain size (determined by the thickness of the filter material) from passing through. This procedure sometimes results in significant resistance for the medium of breathing air, making it harder for the person wearing the mask to breathe. And there is the continual problem caused by the filter effect: the contamination of the filter material's surface with bacteria, viruses, or fungal spores that have been caught in the filter material.

### SUMMARY OF THE INVENTION

Therefore, the present invention aims to provide a half mask for the face, comprising at least one covering area for covering one or both respiratory openings of the person wearing the half mask (meaning their mouth, nose, or both) and at least one fastening area for fastening the half mask to its wearer, specifically to one or more of their head, neck, and throat, that prevents bacteria, viruses, or fungal spores from penetrating the respiratory tract and enables the wearer of the half mask to breathe unimpeded and without resistance.

That objective for a half mask comprising one or more covering areas for covering one or both respiratory openings of the person wearing the half mask and one or more fastening areas for fastening the half mask to its wearer, particularly to one or more of their head, neck, and throat is achieved by providing at least one UV-C disinfection unit for disinfecting the inhaled and exhaled air and at least one device for ensuring that the half mask will tightly fit the contours of its wearer's face. Developments of the invention are defined in the dependent claims.

They enable the creation of a half mask with which the breathing air is not filtered, but disinfected. The UV-C disinfection unit(s) are used to disinfect the inhaled and exhaled air. This is accomplished by irradiating the breathing air with UV-C radiation, which results in excellent disinfection. Because of the one or more devices for ensuring that the half mask will tightly fit the contours of its wearer's face, the half mask can be tightly applied to its wearer's face to keep air from being drawn in or blown out through cracks between the half mask and its wearer's face. This completely shields the breathing air from the environment outside the half mask and ensures that the breathing air is disinfected in both directions during inhalation and exhalation by means of the UV-C disinfection unit(s). It is advantageous to provide for an airway for inhaled and exhaled air in the half mask and arrange the UV-C disinfection unit(s) so that the inhaled and exhaled air streaming through the airway is irradiated with UV-C radiation and thereby disinfected. This means that either the airway always leads through the UV-C radiation or the UV-C radiation is arranged so that it can reliably affect the breathing air streaming through the airway. One or more tubular elements, or one or more tubular sections formed from various components, can be provided as an airway that guides the breathing air through the UV-C disinfection unit.

It is more advantageous if one or more control and/or regulation devices are provided to control and/or regulate the UV-C radiation emitted or emittable from the UV-C disinfection unit, depending on the air quantity within the UV-C disinfection unit. To that end, one or more air mass sensors (to record the air flow rate streaming through the UV-C disinfection unit) and one or more microprocessor control units (to determine the amount of energy necessary to irradiate the air to optimally disinfect it) can be provided. Therefore, the half mask has a function of disinfecting its wearer's respiration, a primary disinfecting element being the tubular element or tubular section through which the breathing air streams. In that element, an optimal energy quantity of UV-C light is irradiated on a predetermined or predeterminable path (specifically, the airway) to effectively change genetic material and therefore render bacteria and viruses dysfunctional. At the same time, permanent exposure to UV-C radiation is avoided, which enables efficient, energy-saving operations.

Studies reveal that ultraviolet radiation with a wavelength between 100 nm and 280 nm is suitable for splitting molecular chains of deoxyribonucleic acid (DNA) or ribonucleic acid (RNA). Because of this quality, irradiating bacteria and viruses with UV-C light or UV-C radiation can prevent them from reproducing. And this in turn means that, if a UV-C disinfection unit is constructively applied in front of the respiratory openings (such as the mouth and nose) of the half mask's wearer, the bacteria and viruses reaching the wearer's respiratory tract will be mostly harmless. The wearer of the half mask will usually be a person, but can also be an animal.

It is advantageous if the device for ensuring that the half mask will tightly fit the contours of its wearer's face is formed from one or more pieces of breathable mesh fabric (at least in the area covering the wearer's respiratory openings). A breathable mesh fabric is a fine cloth consisting of numerous small stiches. Technically speaking, it's a type of grid produced by interconnected threads. The mesh fabric's material is a thin, loosely woven or knitted cloth provided with numerous small holes. Such a mesh fabric can therefore be tightly applied to the contour of the face, fits the facial shape, and enables the half mask to be tightly fit with the surface of its wearer's face. At the same time, it is comfortable to wear, unlike the masks currently available on the market, since no local pressure is exercised, especially at the edges of the half mask on the surface of the facial skin.

It is more advantageous to provide at least one replaceable element for filtering, sealing, or both, in order to prevent the aspiration of air outside the airway (which can be disinfected by one or more UV-C disinfection units) and to prevent droplets from escaping into the surrounding air outside the half mask during exhalation. The replaceable element(s) for filtering, sealing or both prevent the aspiration of air that was not guided through the disinfection stages. During exhalation, the filtering effect of the element(s) for filtering, sealing or both prevents droplets from the exhaled air from being emitted into the surrounding air. For example, the element(s) for filtering, sealing or both can include one or more of the following filter materials: paper filter material, activated carbon element, or HEPA filter element. Depending on the choice of the filter material of the replaceable element(s) for filtering, sealing or both, various degrees of protection can be attained during the filtering of respiratory droplets.

It is more advantageous if the UV-C disinfection unit(s) can comprise one or more bodies (particularly transparent molding) that are transparent for UV-C radiation and have one or more spiral air channels and a number of UV-C LEDs which are used to irradiate the air streaming through the air passage. The transparent bodies or molding for UV-C radiation internally feature the spiral air passage(s), which, for example, can be formed as a spiral tube with a rectangular cross-section. When unwound, it represents the effective length for the UV-C energy input. The UV-C LEDs can be arranged on the outer periphery or the areas of the spiral air passage to irradiate the air stream inside the spiral air passage.

It is advantageous if one or more devices that are lightproof to the outside are provided for receiving the UV-C disinfection unit(s), to protect the wearer of the half mask and its environment from UV-C radiation. In particular, the UV-C disinfection unit(s) can be received or encapsulated in one or more housing units that are lightproof to the outside. Furthermore, to optimize the distribution of the UV-C radiation, one or more reflective coatings of the device(s)—particularly the housing unit(s), the UV-C disinfection unit, and/or in the air passage, or the air passage of the UV-C disinfection unit—can be provided. Such a reflective coating inside the housing element can optimize the distribution of the UV-C radiation over the air passage, particularly the spiral air passage of the UV-C disinfection unit.

It is more advantageous if the UV-C disinfection unit(s) can be or become operated by one or more energy storage devices, such as one or more rechargeable or non-rechargeable batteries. This makes it possible to operate the half mask without providing an electrical cable to connect with an external energy source. Instead, the energy storage device(s) can be applied to the half mask or integrated into it. It is especially advantageous if the control or regulation unit(s) (particularly the microprocessor control unit(s)) monitor the condition or state, such as the state of charge of the energy storage device(s), particularly the imminent end of operation of the UV-C disinfection unit(s). This makes it possible to recharge or change the respective energy storage device, promptly before the operating time of the UV-C disinfection unit ends, due to the low charge level of the energy storage device(s). Furthermore, one or more devices for indicating the state of charge (particularly the aforementioned end of the operating time) can be or become provided to the UV-C disinfection unit, particularly an optical and/or acoustical display device. Preferably, the display device(s) can be controlled by the control or regulation unit. The state of charge of the energy storage device(s) can therefore be monitored by the control or regulation unit, and the current state of charge can be indicated by the optical and/or acoustical display device(s).

It is advantageous if one or more air inlets into the half mask and one or more air outlets out of the mask are provided, wherein the airway that is or should be disinfected extends between them. To prevent the emission of non-disinfected or inadequately disinfected air into the environment of the half mask, one or more valve elements can be or become provided in the area of the half mask's air outlet (or one of the air outlets). For example, the valve element(s) can function electromagnetically, mechanically, or both. It is more advantageous if the valve element(s) can block the airway through the UV-C disinfection unit if it is determined that the air supply stream in the airway is inadequate. If the latter occurs, air purification that is comparable with a standard paper filter mask is guaranteed through the filter and sealing element(s), wherein, due to the provision of the device for form-fitting, tight application of the half mask to the face of its wearer, and the sealing application of the half mask on the face of its wearer, the filter and sealing element(s) are far superior to a typical standard paper filter mask in terms of sealing.

By the combination of a UV-C disinfection unit which is operated by an energy storage device (specifically, a rechargeable or non-rechargeable battery) and whose incoming irradiation on an incoming path, i.e. the airway, is continually controlled or regulated analogously to the air quantity the airway contains and a flat, tightly sealed half mask that also includes a replaceable filter and sealing element the protective effect can be maintained, i.e. if the UV-C disinfection unit fails or if adequate disinfection can no longer be guaranteed.

The half mask is therefore modular in construction and includes the components of the area(s) covering the respiratory opening(s) of a wearer of the half mask, which are provided with one or more devices for ensuring that the half mask will tightly fit the contours of its wearer's face, to apply the half mask in a form-fitting way, tightly to the face of its wearer, in order to apply the half mask around the respiratory opening(s), such as the mouth and nose, the UV-C disinfection unit, and the replaceable filter and sealing element(s). The UV-C disinfection unit(s) are arranged between the area covering one or both respiratory openings and the replaceable filter and sealing element(s) so that the UV-C disinfection unit(s) become or are received between them. To that end, the half mask's area that covers the respiratory opening(s) is arranged on the mask's exterior side and the replaceable filter and sealing element(s) are arranged on its interior side. Since the half mask has a modular design and the UV-C disinfection unit is integrated into it, the half mask's components are easy to clean and disinfect. For example, an air inlet cap can be provided on the exterior side of the half mask to cover the air inlet and a counterpart on the interior side of the air inlet, the air inlet cap and the counterpart being attachable to each other (by providing a magnetic connection, for example). Air enters into the UV-C disinfection unit(s) through the air inlet cap and its counterpart. The counterpart and the UV-C disinfection unit(s) can be or become attached to each other. It is advantageous if an air outlet cap can be arranged on the interior side of the replaceable filter and sealing element(s), which can be or are connected to the air outlet on the exterior side of the replaceable filter and sealing element(s). A magnetic connection can be or become provided to that end. The air outlet is also connectable or connected with the UV-C disinfection unit(s).

To allow the data or operating parameters of the half mask determined by sensor elements to be transmitted to an external data processing unit, one or more transmitters can be provided for transmitting the operating parameters of the half mask (particularly of its UV-C disinfection unit) to the external data processing unit. In particular, the external data processing unit can take on one or more of notifying, warning, and logging functions regarding the half mask, particularly its UV-C disinfection unit. The operating parameters can also be transmitted via a local wireless network to a connected data terminal as an external data processing unit, which can take over the notifying, warning, and logging functions. The operation can take place via a mobile device, such as a mobile telephone (in the manner of an app, for example). These operating parameters and collected data can also be logged (and subsequently transmitted to an EDP system) in the microprocessor control unit(s) provided to monitor the condition of the energy storage device (its state of charge, for example).

Furthermore, one or more sensor elements for detecting medically relevant quantity components of the breathing air can be provided in the half mask (especially in its airway), particularly to record oxygen (O₂), carbon dioxide (CO₂), nitrogen (N₂), acetone, ammoniac, hydrogen (H₂), and other components of the breathing air. The quantity components so determined can be used to recognize illnesses, to adjust athletic training programs to the capability of the face mask's wearer, or both. This means the possibility can be created for notifying the wearer of such a half mask of illnesses early on. And in sports, precise knowledge of the material composition of the breathing air can be helpful in adjusting training programs to the capabilities of a particular individual.

### DETAILED DESCRIPTION OF THE DRAWINGS

For a more detailed explanation of the invention, its features are described in greater detail based on the drawings, wherein

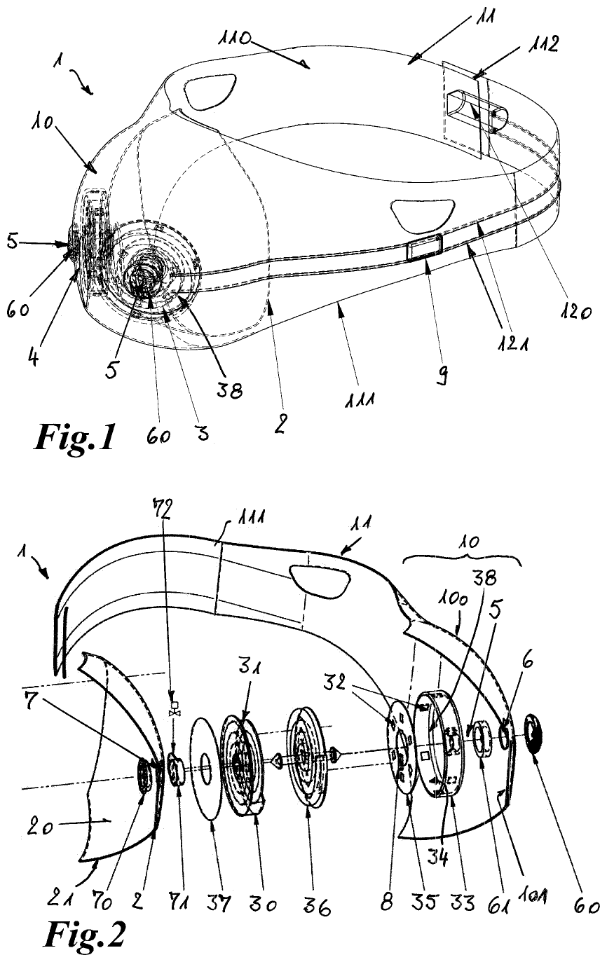

FIG. 1 shows a view of a first embodiment of a half mask according to the invention, outlined in perspective, and

FIG. 2 shows an exploded view, in perspective, of part of a second embodiment of a half mask according to the invention.

### DETAILED DESCRIPTION OF THE INVENTION

FIG. 1 shows a half mask 1, which is provided to be worn on the head of a person or a wearer. For this purpose, it features an area 10 covering the respiratory openings (such as the nose and mouth) of the wearer of the half mask and one or more fastening areas for fastening the half mask to its wearer, particularly to one or more of their head, neck, and throat. The fastening area 11 comprises two ribbon-shaped fastening elements 110 and 111, which are fastened to each other by means of a closure element 112. The closure element 112 can be a Velcro fastener or another closure element, for example.

The covering area of the half mask 1 is designed so that it encloses the respiratory openings (such as the mouth and nose) of the wearer of the half mask 1 by lying flat and tight-fitting on their face. So, it is preferable if the covering area 10 and possibly the fastening area 11 of the half mask 1 consist of an air-permeable mesh fabric that is suitable for being laid on the wearer's facial contours with a tight, close fit. The material of the covering area 10 of the half mask 1 is also used to support additional components of the half mask 1. These components are a replaceable filter and sealing element 2 and (in the embodiment according to FIG. 1) two UV-C disinfection units 3, 4 or (in the embodiment of the half mask 1 according to FIG. 2) only one UV-C disinfection unit 3.

The UV-C disinfection units 3, 4 are used to disinfect the inhaled and exhaled air. They are therefore arranged in the airway 5 of the breathing air. This is given, wherein the aspiration of air outside the airway 5—air that was not guided through the disinfection stages of the UV-C disinfection unit(s)—is prevented by means of the replaceable filter and sealing element 2. During exhalation, the filtering effect of the filter and sealing element 2 prevents droplets from the air exhaled by the half mask's wearer from being emitted into the surrounding air. This replaceable filter and sealing element 2 can be a simple filter, an active carbon element, or a HEPA filter element, wherein various degrees of protection can be attained during the filtering of respiratory droplets depending on the choice of the filter and sealing element.

The airway 5 extends between an air inlet 6 and an air outlet 7. The air inlet is formed by one or more openings in the covering area 10, as can be better inferred from FIG. 2 in particular. Two such openings (air inlets 6) are provided in the embodiment of the half mask according to FIG. 1, although, according to FIG. 2, merely one such opening or one such air inlet 6 is provided. The air inlet 6 is covered by an air inlet cap 60 on the exterior side 100 of the covering area 10 of the half mask 1. An air inlet counterpart 61 to the air inlet cap 60 is arranged on the interior side 101 of the covering area 10. The air inlet cap 60 and its air inlet counterpart 61 are connected with each other (magnetically, for example). Therefore, they adhere to each other on the exterior side 100 and the interior side 101 of the covering area 10, whose material between them receives them.

The air outlet 7 is formed by one or more openings in the replaceable filter and sealing element 2. On the interior side 20 of the replaceable filter and sealing element 2, an air outlet cap 70 is arranged, and on the exterior side 21 of the replaceable filter and sealing element 2, a corresponding air outlet counterpart 71 is arranged, which is connectable or connected to the air outlet cap 70. A magnetic connection is suitable to that end as well. The air outlet cap 70 and its air outlet counterpart 71 receive the replaceable filter and sealing element 2 between them, as shown in FIG. 2.

The UV-C disinfection unit(s) 3 or 4 are arranged between the two counterparts 61, 71 and in particular connected with them. The airway 5 therefore leads through the air inlet cap(s) 60, the air inlet(s) 6, their air inlet counterpart(s) 61, the respective UV-C disinfection unit 3, 4 and the air outlet counterpart(s) 71, the air outlet(s) 7, and the air outlet cap(s) 70.

One or more openings can be arranged in the covering area 10 of the half mask 1 and the replaceable filter and sealing element 2 to function as air inlet 6 and air outlet 7. To optimize the form of the half mask 1, multiple UV-C disinfection units 3, 4 (as shown in FIG. 1) can be provided, each with air inlet caps as outer coverings, instead of only one UV-C disinfection unit 3 as shown in FIG. 2.

The main components of the UV-C disinfection unit 3 or 4 consist of a mold 30 that is transparent for UV-C light. This is shown in FIG. 1 as a cylindrical mold, but can also feature another design. As can be better seen in FIG. 2, the mold 30 has a spiral shape in the interior and features a spiral air passage 31 emanating from its center, which can be formed as a rectangular tube in cross section, for example. The spiral air passage 31 represents the effective length for the energy input of the UV-C radiation when unwound.

The molding 30 is received or encapsulated, lightproof, in a housing 33, to avoid an emission of UV-C radiation from the housing 33 and therefore guarantees protection from the genetic-material-altering effect of the UV-C radiation. A number of UV-C LEDs 32 are arranged on the interior side of the housing 33, on its outer periphery 34, and on its surface, which is shown in FIG. 2 as a disc-shaped housing cover 35. These are used to irradiate an air stream 36 during its passage through the molding 30 or its spiral air passage 31. The UV-C LEDs 32 are arranged in the housing 33 toward the side of the connection with the air inlet counterpart 61. The housing 33 also features a housing cover 37 toward the air outlet counterpart 71. The air outlet counterpart 71 is connected with it.

The interior side 38 of the housing 33 can be provided with a reflective coating to optimize the distribution of the UV-C radiation along the airway in the spiral air passage 31.

To supply the UV-C LEDs 32 with energy, an energy storage device 120 in the form of one or more rechargeable or non-rechargeable batteries is provided and fastened to the half mask 1. As shown in FIG. 1, the energy storage device 120 in the area of the enclosure element 112 is fastened to one or more ribbon-shaped fastening elements 110, 111. Between the UV-C disinfection unit(s) 3, 4 and the energy storage device(s) 120, electrical lines 121 are provided that extend along the covering area 10 and the ribbon-shaped fastening element 111 in the embodiment of the half mask 1 shown in FIG. 1.

As shown in FIG. 2, an air mass sensor 8 is arranged in the airway 5 in this embodiment variant, that air mass sensor encompassing the flow quantity of the air that streams through the UV-C disinfection unit 3 or units 3 and 4. A microprocessor control unit 9 or a microprocessor-controlled control loop calculates the amount of energy which is needed for the disinfection and with which the air quantity within the UV-C disinfection unit(s) 3 or 4 is to be irradiated to achieve an optimal effect. Through this procedure, permanent exposure can be avoided, which allows for efficient operations with an energy storage device 120 in the form of a rechargeable battery pack, for example. The microprocessor control unit 9 is arranged along both electrical lines 121 as shown in FIG. 1.

To prevent a possible emission of non-disinfected or inadequately disinfected air into the environment, a valve element 72 is integrated into the embodiment of the half mask 1 according to FIG. 2, particularly in the form of an electromagnetic and/or mechanically activated valve element that automatically blocks the air passage 31 through the UV-C disinfection unit 3 or 4 if the power supply is inadequate. If this occurs (meaning, if no disinfection of the air takes place), an air filtration (and therefore an air purification) comparable to that of a standard paper filter mask is guaranteed by means of the replaceable filter and sealing element 2. Regarding the sealing of the masks when they are applied to the face of their wearer, the half mask 1 is significantly superior to the well-known standard paper marks.

The modular construction and simple integration of the disinfecting element make it possible for its components to be cleaned and disinfected easily. By loosening the respective connection between the air inlet 60 and the air inlet counterpart 61 or the air outlet cap 70 and the air outlet counterpart 71, all components of the half mask 1 can be separated from each other; in particular, the replaceable filter and sealing element 2 can be replaced.

By permanently monitoring the condition (especially the state of charge) of the energy storage device 120 as that of the rechargeable battery status, any imminent ending of the operating time of the UV-C disinfection unit(s) 3 or 4 can be optically and acoustically signaled by providing an optical and/or acoustic display device 90.

The operating parameters determined can also be transmitted via a wireless network (particularly a local one) to a connected data terminal as an external data processing unit connectable or connected to it, which can take over the notifying, warning, and logging functions. The operation can take place via a mobile device, such as a mobile telephone (in the manner of an app). And the operating parameters or recorded data can also be logged and subsequently transmitted to an EDP system by means of the microprocessor control unit 9.

Through the defined or definable geometry of the spiral air passage 31 of the UV-C disinfection unit(s) 3 or 4, a sensor element (not shown in FIG. 1 or 2) can be or become provided to determine various medicinally relevant quantity components of the breathing air and integrated into the UV-C disinfection unit(s) 3 or 4. For example, O₂, CO₂, N₂, acetone, ammoniac, H₂, and other components of the breathing air can be determined as medicinally relevant by the sensor element(s). The combination with the data of the existing air mass sensor 8 makes it possible to notify the wearer of the half mask 1 of illnesses early on. And in sports as well, precise knowledge of the material composition of the breathing air can be helpful in adjusting training programs to the capabilities of a particular individual, for example.

Besides the embodiments of half masks described in the foregoing and shown in the figures, numerous other formations are possible, particularly any desired combination of the aforementioned features, wherein the face mask entails one or more covering areas to cover one or both respiratory openings of the person wearing the half mask, one or more fastening areas to fasten the half mask to the face of its wearer, particularly one or more of their head, neck, and throat, one or more UV-C disinfection units to disinfect the inhaled air, and one or more devices for applying the half mask to the face of the person wearing it in a tight, form-fitting manner.

### LIST OF REFERENCE NUMBERS

1 Half Mask for the Face
2 Filter and sealing element
3 UV-C disinfection unit
4 UV-C disinfection unit
5 Airway
6 Air inlet
7 Air outlet
8 Air mass sensor
9 Microprocessor control unit
10 Covering area
11 Fastening area
20 Interior side/inner surface
21 Exterior side/outer surface
30 Molding
31 Spiral air passage
32 UV-C LED
33 Housing
34 Outer perimeter/outer periphery
35 Flat/disc-shaped housing cover
36 Air stream
37 Housing cover
38 Interior side of the housing
60 Air inlet cap
61 Air inlet counterpart element
70 Air outlet cap
71 Air outlet counterpart element
72 Valve element
90 Display device
100 Exterior side/outer surface
101 Interior side/inner surface
110 Ribbon-shaped fastening element
111 Ribbon-shaped fastening element
112 Closure element
120 Energy storage device (rechargeable or non-rechargeable battery)
121 Electrical line

What is claimed is:

1. A half mask for the face, comprising:
one or more covering areas for covering one or both respiratory openings of a person wearing the half mask and one or more fastening areas for fastening the half mask to its wearer, to one or more of their head, neck, and throat,
wherein one or more UV-C disinfection units for disinfecting inhaled air and exhaled air and one or more devices for ensuring that the half mask will tightly fit the contours of its wearer's face are provided.

2. The half mask in accordance with claim 1, wherein an airway for inhaled and exhaled air is provided in the half mask and the one or more UV-C disinfection unit are arranged so that the inhaled and exhaled air streaming through the airway is irradiated with UV-C radiation and thereby disinfected.

3. The half mask in accordance with claim 2, wherein one or more control and/or regulation devices are provided to control and/or regulate the UV-C radiation emitted or emittable from the UV-C disinfection unit depending on the air quantity within the UV-C disinfection unit.

4. The half mask in accordance with claim 3, wherein one or more air mass sensors are provided to record the air flow rate streaming through the UV-C disinfection unit and the control and/or regulation device(s) entail one or more microprocessor control units to determine the amount of energy necessary to irradiate the air to optimally disinfect it.

5. The half mask in accordance with claim 1, wherein a device for the form-fitting, tight application of the half mask to the face of its wearer can be arranged and the half mask is formed from one or more breathable mesh fabrics at least in the area covering the wearer's respiratory openings.

6. The half mask in accordance with claim 2, wherein at least one replaceable filter and/or sealing element is provided to prevent the aspiration of air outside the airway which can be disinfected by one or more UV-C disinfection units and to prevent droplets from escaping into the surrounding air outside the half mask during exhalation.

7. The half mask in accordance with claim 6, wherein the replaceable filter and/or sealing element(s) include one or more of the following filter materials: paper filter material, activated carbon element, or HEPA filter element.

8. The half mask in accordance with claim 1, wherein the UV-C disinfection unit(s) can comprise one or more bodies that are transparent for UV-C radiation and a number of UV-C LEDs which are used to irradiate the air streaming through the air passage.

9. The half mask in accordance with claim 1, wherein one or more devices that are lightproof to the outside are provided for receiving the UV-C disinfection unit(s), to protect the wearer of the half mask and its environment from UV-C radiation.

10. The half mask in accordance with claim 9, wherein one or more reflective coatings of the device(s) are provided to optimize the distribution of the UV-C radiation.

11. The half mask in accordance with claim 1, wherein one or more UV-C disinfection units are operable or operated by one or more energy storage devices.

12. The half mask in accordance with claim 11, wherein microprocessor control unit(s) monitor the state of charge of the energy storage device(s) and at least one device is provided to display the state of charge of the UV-C disinfection unit(s) and the display device(s) are controllable or controlled by the control and/or regulation unit.

13. The half mask in accordance with claim 1, wherein to prevent the emission of non-disinfected or inadequately disinfected air into the environment of the half mask, one or more valve elements are provided in the area of an air outlet of the half mask, wherein the valve element(s) block the airway through the UV-C disinfection unit(s) if it is determined that the air supply stream in the airway is inadequate.

14. The half mask in accordance with claim 1, wherein one or more transmission devices are provided for transmitting operating parameters of the half mask UV-C disinfection unit(s), to an external data processing unit, that takes on one or more of notification, warning, and logging functions regarding the half mask UV-C disinfection unit(s).

15. The half mask in accordance with claim 1, wherein one or more sensor elements for detecting medically relevant quantity components of the breathing air can be provided in the half mask to record oxygen, carbon dioxide, nitrogen, acetone, ammoniac, hydrogen, and other components of the breathing air, to identify illnesses and/or to adjust athletic training programs to the capability of the person wearing the half mask.

16. The half mask in accordance with claim 8, wherein the one or more bodies are transparent moldings that have one or more spiral air channels.

17. The half mask in accordance with claim 9, wherein the UV-C disinfection unit(s) are received or encapsulated in the housing unit(s) in a manner that is lightproof to the outside.

18. The half mask in accordance with claim 10, wherein one or more reflective coatings are coatings of the housing, the UC-C disinfection unit(s), or the air passage of the UV-C disinfection unit(s), and/or in the air passage.

19. The half mask in accordance with claim 11, wherein the one or more energy storage devices are one or more rechargeable batteries and/or one or more batteries.

20. The half mask in accordance with claim 12, wherein the at least one device for displaying the state of charge is an optical or acoustic display device.

21. The half mask in accordance with claim 13, wherein the valve elements are electromagnetic and/or mechanical valve elements.
